(19) United States
(12) Patent Application Publication
(10) Pub. No.: US 2024/0315648 A1
(43) Pub. Date: Sep. 26, 2024

(54) Seizure Forecasting in Subsutaneous Electroencephalography Data Using Machine Learning

(71) Applicants: Mayo Foundation for Medical Education and Research, Rochester, MN (US); King's College London, London (GB)

(72) Inventors: Benjamin H. Brinkman, Byron, MN (US); Mona Nasseri, Ponte Vedra Beach, FL (US); Tal Pal Attia, Rochester, MN (US); Gregory A. Worrell, Rochester, MN (US); Mark P. Richardson, Croydon (GB); Pedro Viana, London (GB)

(21) Appl. No.: 18/579,582

(22) PCT Filed: Jul. 14, 2022
(86) PCT No.: PCT/US2022/037157

§ 371 Date: Jan. 16, 2024

Related U.S. Application Data
(60) Provisional application No. 63/222,867, filed on Jul. 16, 2021.

(51) Int. Cl. A61B 5/00; A61B 5/384; G06N 3/0442; G06N 3/08

(52) U.S. Cl. CPC A61B 5/7275; A61B 5/0006; A61B 5/0022; A61B 5/384; A61B 5/4094; A61B 5/7267; A61B 5/7405; A61B 5/742; A61B 5/746; A61B 5/7475; G06N 3/0442; G06N 3/08

(57) ABSTRACT

Seizure onset can be forecast in subjects from subcutaneous electroencephalography (EEG) data input to a trained machine learning algorithm. A multi-stage training process is used to train the machine learning algorithm. A first stage of the training process is implemented on scalp-recorded EEG data. A second stage of the training process may be implemented on subcutaneously recorded EEG data.

### BACKGROUND

Despite progress in medical, surgical, and neuromodulation therapies for epilepsy, many patients continue to experience seizures. Subcutaneous EEG is an emerging method for long-term monitoring of patients with epilepsy. Electrodes are inserted under the skin surface surgically, and EEG is recorded and telemetered to an external storage device.

### SUMMARY OF THE DISCLOSURE

The present disclosure addresses the aforementioned drawbacks by providing a method for predicting a seizure onset in electroencephalography (EEG) measurement data recorded with a subcutaneous EEG measurement device. Subcutaneous EEG measurement data are recorded with the subcutaneous EEG device, where the subcutaneous EEG measurement data includes EEG signals measured subcutaneously from a subject. A trained machine learning algorithm is accessed with a computer system, where the trained machine learning algorithm has been trained on training data in order to predict a likelihood of seizure onset occurring within the EEG signals contained in the subcutaneous EEG measurement data. The subcutaneous EEG measurement data are transmitted from the subcutaneous EEG device to the computer system and applied to the trained machine learning algorithm with the computer system, generating output as an indication of seizure onset occurring in the subcutaneous EEG measurement data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 illustrates an example neural network architecture for forecasting seizure onset from subcutaneously recorded electroencephalography (EEG) signals. The example neural network architecture implements a three-layer long-short-term-memory (LSTM) network to train an initial classifier, which can be retrained in transfer learning.

FIG. 2 is a flowchart setting forth the steps of an example method for forecasting, detecting, classifying, or otherwise monitoring seizures based on subcutaneous EEG data.

FIG. 3 is a flowchart setting forth the steps of an example method for training a machine learning algorithm using a multi-stage training process in order to forecast, detect, classify, or otherwise monitor seizures based on subcutaneous EEG data.

FIG. 4 is a block diagram of an example system for forecasting seizure onset based on subcutaneous EEG data.

FIG. 5 is a block diagram of example components that can implement the system shown in FIG. 4.

### DETAILED DESCRIPTION

Described here are systems and methods for forecasting epileptic and other seizures in patients using subcutaneous electroencephalography (“EEG”) recordings applied to a trained machine learning algorithm, such as a trained neural network. The systems and methods described in the present disclosure address and overcome limitations of previous seizure prediction methods by operating on EEG data measured using subcutaneous electrodes and using a multi-stage training process. The machine learning algorithm(s) can advantageously be trained using a cross-subject training/testing approach, such that cross-subject classification can be implemented for forecasting epileptic and other seizures in patients.

The seizure forecasting algorithms described in the present disclosure can be run at fixed intervals on the most recent subcutaneous EEG data measured from a user, and the algorithms will classify the data with a seizure classification, such as pre-seizure or baseline EEG. If the subcutaneous EEG data in a given epoch are classified as pre-seizure, an alert can be generated and presented to the user as an audible tone, vibration, text message to a caregiver, or other method. The user may use this information to modify activities, take a physician-prescribed medication, or ask a caregiver for help. The user may also increase stimulation levels in an existing neuromodulation device, or have the stimulation levels automatically adjusted based on the classification of the EEG data.

As a non-limiting example, a first training process can be implemented on EEG data obtained from scalp-recorded EEG measurements. A second training process can be implemented on subcutaneous EEG data.

Using the input data and two-stage training process, which incorporates data from both scalp-recorded and subcutaneously recorded EEG data, the systems and methods described in the present disclosure improve upon previous methods for forecasting seizures.

Described here are neural network architectures for analyzing time-series signals to forecast seizure onset in data recorded from subcutaneous EEG measurements. Transfer learning is used to adapt a classifier trained on scalp-recorded EEG signals to analyze subcutaneous EEG data. Deep learning is a powerful technique with great potential, but some challenges have to be overcome to apply it in practice. Abundant amounts of data are required to train deep learning algorithms since they learn progressively. Data availability for these algorithms for some applications may be limited, such as in epilepsy, where numbers of study subjects are limited, and independent verification of seizures is needed for reliable training and testing subcutaneous EEG data. Another challenge of deep learning is the massive amounts of processing power required for training, and even with multi-core high-performance graphics processing units, training can be time consuming. To overcome these challenges, the systems and methods described in the present disclosure make use of available scalp-recorded EEG data and use this data as an initial training set in a multi-level transfer learning approach. Using transfer learning to retrain the last layer(s) of the algorithm can reduce the training time and improve the classifier's accuracy.

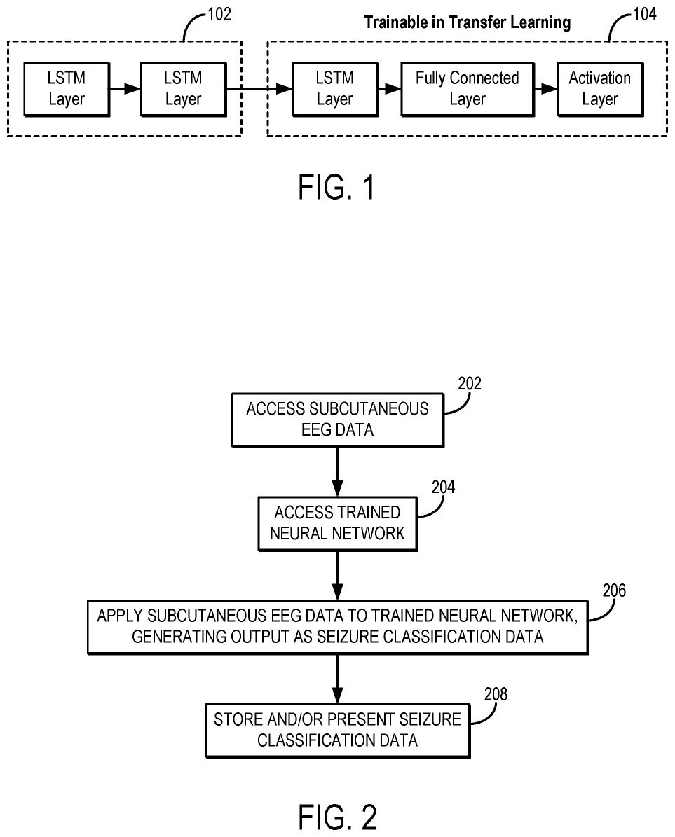

An example three-layer long-short-term-memory (“LSTM”) neural network algorithm that can be implemented by the systems and methods described in the present disclosure is shown in FIG. 1. A unidirectional LSTM algorithm, in which the LSTM unit processes data segment time instances sequentially in one direction, is used in this example. The main component of the LSTM network is the hidden layer, which includes the memory cells. Each memory cell has three gates (forget, input, output) to control the cell behavior across time. The cell gates allow the network to detect dependencies in the stream of input data. The input to the LSTM network is in the form of a sequence input layer, and after all the LSTM layers, there is a fully connected layer and an output layer to generate the classification output using a sigmoid, or other suitable, activation function.

In one non-limiting example, three consecutively connected LSTM layers (3 LSTM) and one dropout layer after each LSTM layer can be used. As one example, the LSTM layers can include 100-200 hidden layers and the dropout layers can have a rate of 0.2. The dropout layer randomly sets input units to zero at each step during training to reduce overfitting. The network can be set to train for a selected number of epochs (e.g., 200 epochs with a batch size of 100). Additionally or alternatively, early stopping can be used to halt training when there is little to no significant improvement in the results.

In another non-limiting example, five consecutively connected LSTM layers and one dropout layer after each LSTM layer can be used. As an example, the LSTM layers can include fewer hidden layers than used in a 3 LSTM architecture. For instance, the LSTM layers can include 25 hidden layers, or the like.

In yet another non-limiting example, a bidirectional LSTM (“BiLSTM”) architecture can be used. For instance, a BiLSTM architecture with two bidirectional LSTM layers can be used. As a non-limiting example, each BiLSTM layer can have 10 hidden layers, or the like.

The LSTM architecture can also be used for transfer learning, in which a model pre-trained on scalp-recorded EEG data dataset is applied. The first two LSTM layers of the pre-trained network, noted as section 102 in FIG. 1, can be considered non-trainable, and the rest of the network, noted as section 104 in FIG. 1, can be set as the trainable part of the system.

Referring now to FIG. 2, a flowchart is illustrated as setting forth the steps of an example method for forecasting seizure onset in subcutaneous EEG measurement data using a suitably trained neural network or other machine learning algorithm.

The method includes accessing subcutaneous EEG measurement data with a computer system, as indicated at step 202. Accessing the subcutaneous EEG measurement data may include retrieving such data from a memory or other suitable data storage device or medium. Alternatively, accessing the subcutaneous EEG measurement device data may include acquiring such data with a subcutaneous EEG device and transferring or otherwise communicating the data to the computer system, which may be a desktop computer, a laptop computer, a smartphone, a tablet computer, a wearable computer, a server computer, a virtual machine being executed by a physical computing device, and so on. For instance, the subcutaneous EEG data can be wirelessly transmitted (e.g., via a Bluetooth link, or the like) from the subcutaneous EEG device to the computer system, which in some non-limiting examples may be a smartphone.

A trained neural network (or other suitable machine learning algorithm) is then accessed with the computer system, as indicated at step 204. Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

In general, the neural network is trained, or has been trained, on training data in order to detect seizures based on subcutaneous EEG measurement data obtained from a subject having a subcutaneous EEG device that measures suitable EEG signal data. The probability of seizure onset can thus be detected or otherwise determined based on the EEG measurement data.

As a non-limiting example, the neural network can include a multi-layer LSTM neural network that has been trained to identify pre-seizure EEG segments in subcutaneous EEG recordings. An example network architecture is shown in FIG. 1 and described above. In this example, the algorithm uses three LSTM layers: however, the network may also include more layers, such as four or five LSTM layers. In a non-limiting example, each LSTM layer can include several hundred hidden layers (e.g., 100-200 hidden layers).

Thus, as one example, the neural network architecture can include three LSTM layers with two input channels for filtered subcutaneous EEG data, and two FFT channels. As another example, the neural network can take two filtered down-sampled channels, 2 FFT channels, and one time-of-day channel. In these instances, the neural network architecture may include ten LSTM layers with 25 units each, two bidirectional LSTM (“BiLSTM”) layers with 20 units each, or a simple recurrent neural network (“RNN”) architecture. In some embodiments, the neural network can include other recurrent neural network architectures, such as recurrent neural network architectures that include one or more gated recurrent unit (“GRU”) layers. The neural network may also include one or more convolutional layers. In some configurations, the neural network may include fully connected layers.

The subcutaneous EEG measurement data are then input to the trained neural network, generating output as seizure classification data, as indicated at step 206. In some instances, the subcutaneous EEG data can be pre-processed before being applied to the trained neural network. As a non-limiting example, the subcutaneous EEG data can be segmented into epochs (e.g., one-minute epochs) and pre-processed with per-segment mean subtraction, low pass filtering (e.g., at 25 Hz), and fast Fourier transform (“FFT”) for each channel. The subcutaneous EEG data may in some instances also be normalized (e.g., z-score). To account for circadian and multi-day periodicities in seizure risk, the time of day and/or the time since the last seizure event can be encoded as an additional channel input. Additionally or alternatively, a signal quality metric can be added as an additional channel or as a discrete input to enable the algorithm to compensate for noisy or artifact-corrupted data.

The seizure classification data may include a classification of all or a portion of the EEG measurement data as corresponding to pre-seizure EEG signals or baseline EEG signals. For instance, the seizure classification data may indicate the probability for the subject to experience a seizure within a particular time frame (e.g., within the next 60-90 minutes), or that the subject is presently experiencing symptoms of a seizure event. As another example, the trained neural network may be trained to implement automatic pattern recognition to generate seizure classification data that classify whether signals within the subcutaneous EEG measurement data correspond to, or otherwise indicate the presence of, an imminent or present seizure.

The seizure classification data generated by inputting the subcutaneous EEG data to the trained neural network can then be displayed or otherwise presented to a user, stored for later use or further processing, or both, as indicated at step 208. For instance, the seizure classification data can be presented to the user as an auditory, visual, or haptic alarm indicating that a seizure event is imminent or otherwise predicted to occur within a duration of time (e.g., within the next 60-90 minutes), or presented as such an alarm to indicate to others that the user is experiencing or about to experience a seizure.

In some embodiments, the user can provide feedback indicating false alarms (e.g., a false positive, in which no actual seizure even occurred after the alert) and/or missed seizure events (e.g., a false negative, in which a seizure event occurred, but no alarm was generated). This feedback data can be used to retrain or adapt the neural network, or otherwise adjust a threshold for generating an alarm. For instance, active learning can be used to retrain the neural network based on the feedback data, and/or the threshold for generating an alarm can be adjusted after a missed seizure.

The user may provide the feedback data via a computer system in communication with the subcutaneous EEG system. For example, the alarm may be presented to the user via a mobile device (e.g., a smart phone, a smart watch) and in response to the alarm the user may also be presented with a user interface to provide feedback on whether the alarm is a false alarm. The mobile device can receive the feedback data from the user and either provide retraining of the neural network on the mobile device, or can transmit the feedback data to another computer system (e.g., a server) where the neural network can be retrained and/or adapted based on the feedback data. The retrained and/or adapted neural network parameters can then be provided again to the computer system to update the trained neural network. Additionally or alternatively, the feedback data can be used to adjust a threshold for generating an alert.

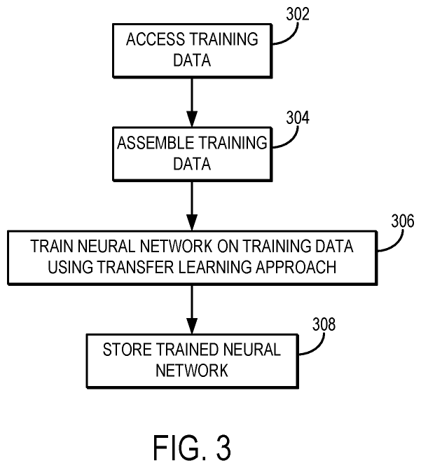

Referring now to FIG. 3, a flowchart is illustrated as setting forth the steps of an example method for training one or more neural networks (or other suitable machine learning algorithms) on training data, such that the one or more neural networks are trained to receive input as subcutaneous EEG data in order to generate output as seizure classification data.

In general, the neural network(s) can implement any number of different neural network architectures. For instance, the neural network(s) could implement a CNN, an RNN, an LSTM network, a neural network having at least one GRU layer, and so on. In some instances, the neural network(s) may implement deep learning. Alternatively, the neural network(s) could be replaced with other suitable machine learning algorithms, such as those based on supervised learning, unsupervised learning, deep learning, ensemble learning, dimensionality reduction, and so on. In some embodiments, active learning may be used when training the neural network(s).

The method includes accessing training data with a computer system, as indicated at step 302. Accessing the training data may include retrieving such data from a memory or other suitable data storage device or medium. Alternatively, accessing the training data may include acquiring such data with a suitable measurement system (e.g., scalp-based EEG electrodes, subcutaneous EEG electrodes) and transferring or otherwise communicating the data to the computer system, which may be a part of the measurement system.

In general, the training data can include subcutaneous EEG recordings acquired from one or more subjects over a duration of time. In some instances, the subcutaneous EEG recordings in the training data can be acquired over a duration of time spanning multiple months. As a non-limiting example, subcutaneous EEG data can include EEG signals measured using two bipolar channels from electrodes positioned over the temporal lobe. The training data can be collected from a cohort of subjects, such as subjects who suffer from epilepsy. The training data can also include scalp-recorded EEG data recorded from the same subjects, or from different subjects, than the subcutaneous EEG data.

Additionally or alternatively, the method can include assembling the training data, as indicated at step 304. This step may include assembling the subcutaneous and/or scalp EEG data into the appropriate data structure(s) on which the neural network or other machine learning algorithm can be trained.

As one non-limiting example, assembling the training data may include assembling the EEG data into a selected data structure. For instance, the EEG data can be segmented into epochs and preprocessed. As an example, the EEG data can be segmented into one-minute epochs. Preprocessing the EEG data can include per-segment mean subtraction, low pass filtering (e.g., low pass filtering at 25 Hz), and applying a fast Fourier transform (“FFT”) to each channel.

Finally, the entire data set can be normalized (e.g., using z-score normalization). To account for circadian and multi-day periodicities in seizure risk the time of day and/or the time since the last seizure event can be encoded as an additional channel input. In addition a signal quality metric can be added as an additional channel or as a discrete input to enable the algorithm to compensate for noisy or artifact-corrupted data.

Assembling the training data may include applying one or more data augmentation processes, such as generating cloned data from the EEG data. Cloned data can be generated by making copies of the EEG data while altering or modifying each copy of the respective EEG data. For instance, cloned data can be generated using data augmentation techniques, such as adding noise to the original data, performing a deformable transformation (e.g., translation, rotation, both) on the original data, smoothing the original data, applying a random geometric perturbation to the original data, combinations thereof, and so on. As one non-limiting example, additional training data can be generated by generating noise-added copies of pre-ictal data segments.

As a non-limiting example, data can be labeled as preictal or interictal epochs as follows: preictal data segments can be defined one hour with a set-back of five minutes before seizure onset for lead seizures, which can be defined as seizures separated from preceding seizures by at least four hours. Clustered (i.e., nonlead) seizures can be excluded from the analysis. Interictal data segments can be identified from seizure-free periods at least one day apart from any seizure. In a leave-one-patient-out analysis, a classifier can be tested on each subject in turns using all other subjects' data as a training set.

In one example implementation, for inclusion as training and testing data, the training data may include a minimum of three 60-minute preictal epochs, the testing data may include a minimum of four 60-minute preictal epochs, the training data may include a total of interictal segments at least three times the total of preictal segments, and the testing data may include at least as many interictal segments as preictal segments.

One or more neural networks (or other suitable machine learning algorithms) are trained on the training data, as indicated at step 306. In general, the neural network can be trained by optimizing network parameters (e.g., weights, biases, or both) based on the three-stage training process described above.

Training a neural network may include initializing the neural network, such as by computing, estimating, or otherwise selecting initial network parameters (e.g., weights, biases, or both). Training data can then be input to the initialized neural network, generating output data. The quality of the output data can be evaluated, such as by passing the output data to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. When the error has been minimized (e.g., by determining whether an error threshold or other stopping criterion has been satisfied), the current neural network and its associated network parameters represent the trained neural network.

In a non-limiting example, a neural network, classifier, or other machine learning algorithm can be first trained on the scalp-recorded EEG data contained in the training data. For instance, scalp-recorded EEG data measured from electrodes overlying the same brain area as the subcutaneous electrodes can be used to pre-train the neural network or other machine learning algorithm. As a non-limiting example, from the international 10-20 system: recordings from F7, T7, P7 on the left, and F8, T8, P8 on the right can be used. Additionally or alternatively, recordings from T9/T10 and P9/P10 electrodes can be used. Signals can be re-referenced to the T7/T8 or comparable channel, and used to pre-train the neural network algorithm, or other machine learning algorithm, on an abundant dataset with similar characteristics to the subcutaneous EEG data.

In the example network described above, the last LSTM and dense layers of the initial algorithm can then be retrained using the subcutaneous EEG data contained in the training data. The effect of this retraining phase is to produce a neural network, or other machine learning algorithm, that is well-tuned to predict the onset of seizure activity from subcutaneous EEG measurements. Consecutive data epochs (e.g., 10-second data epochs, 30-second data epochs, 60-second data epochs) can be extracted and preprocessed. The normalized training dataset can be standardized by subtracting the population mean from each value and dividing by the population standard deviation. The training dataset mean and standard deviation can be used for standardizing the test dataset. Start and end times of seizures can be determined (e.g., according to video-iEEG or scalp EEG monitoring), and seizures extracted from the subcutaneous EEG data based on these timestamps.

The one or more trained neural networks are then stored for later use, as indicated at step 308. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

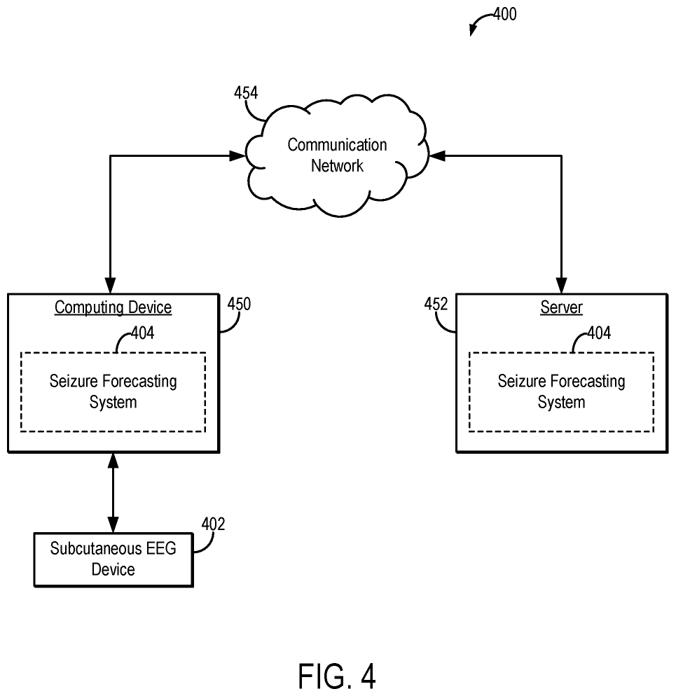

Referring now to FIG. 4, an example of a system 400 for forecasting seizure onset based on subcutaneous EEG data collected by a subcutaneous EEG device, in accordance with some embodiments of the systems and methods described in the present disclosure is shown. As shown in FIG. 4, a computing device 450 can receive one or more types of data (e.g., subcutaneous EEG data) from a subcutaneous EEG device 402 implanted within a subject. In some embodiments, computing device 450 can execute at least a portion of a seizure forecasting system 404 to forecast seizure onset from data received from the subcutaneous EEG device 402. The subcutaneous EEG device 402 can be a unilateral device (i.e., a device with one or more subcutaneous electrodes on only one side of the subject's head), a bilateral device (i.e., a device with one or more subcutaneous electrodes on each side of the subject's head), or two unilateral devices that are placed bilaterally (i.e., one unilateral device on one side of the subject's head, and a second unilateral device on the opposite side of the subject's head).

Additionally or alternatively, in some embodiments, the computing device 450 can communicate information about data received from the subcutaneous EEG device 402 to a server 452 over a communication network 454, which can execute at least a portion of the seizure forecasting system 404. In such embodiments, the server 452 can return information to the computing device 450 (and/or any other suitable computing device) indicative of an output of the seizure forecasting system 404.

In some embodiments, computing device 450 and/or server 452 can be any suitable computing device or combination of devices, such as a desktop computer, a laptop computer, a smartphone, a tablet computer, a wearable computer, a server computer, a virtual machine being executed by a physical computing device, and so on.

In some embodiments, the subcutaneous EEG device 402 can be local to computing device 450. For example, the subcutaneous EEG device 402 can be incorporated with computing device 450 (e.g., computing device 450 can be configured as part of a device for capturing, recording, and/or storing subcutaneous EEG device data). As another example, subcutaneous EEG device 402 can be connected to computing device 450 by a cable, a direct wireless link, and so on. Additionally or alternatively, in some embodiments, subcutaneous EEG device 402 can be located locally and/or remotely from computing device 450, and can communicate data to computing device 450 (and/or server 452) via a communication network (e.g., communication network 454).

In some embodiments, communication network 454 can be any suitable communication network or combination of communication networks. For example, communication network 454 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 454 can be a local area network, a wide area network, a public network (e.g., the Internet), a private or semi-private network (e.g., a corporate or university intranet), any other suitable type of network, or any suitable combination of networks. Communications links shown in FIG. 4 can each be any suitable communications link or combination of communications links, such as wired links, fiber optic links, Wi-Fi links, Bluetooth links, cellular links, and so on.

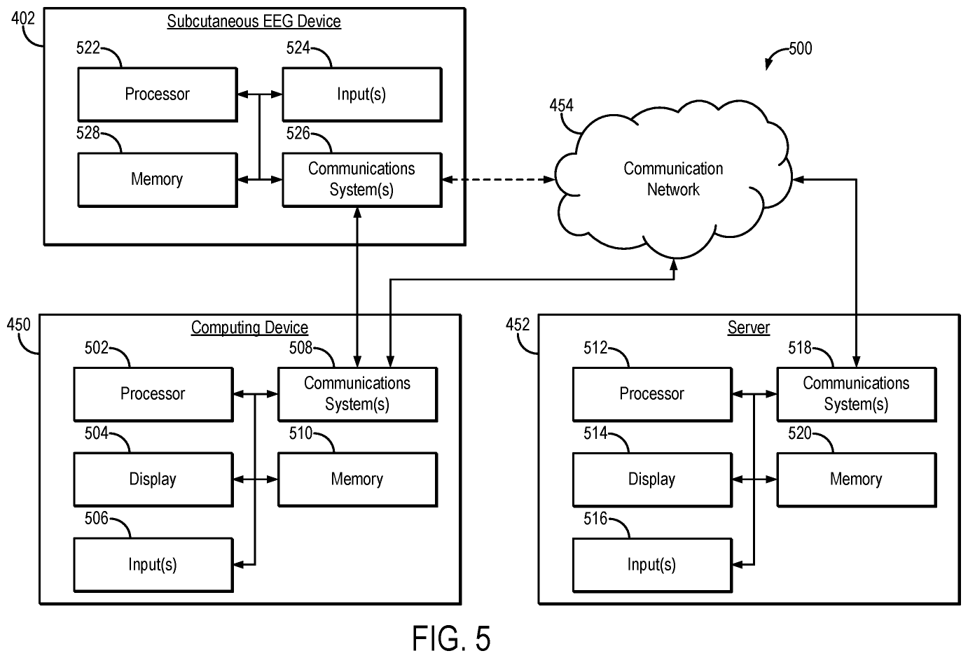

Referring now to FIG. 5, an example of hardware 500 that can be used to implement subcutaneous EEG device 402, computing device 450, and server 452 in accordance with some embodiments of the systems and methods described in the present disclosure is shown. As shown in FIG. 5, in some embodiments, computing device 450 can include a processor 502, a display 504, one or more inputs 506, one or more communication systems 508, and/or memory 510. In some embodiments, processor 502 can be any suitable hardware processor or combination of processors, such as a central processing unit (“CPU”), a graphics processing unit (“GPU”), and so on. In some embodiments, display 504 can include any suitable display devices, such as a computer monitor, a touchscreen, a television, and so on. In some embodiments, inputs 506 can include any suitable input devices and/or sensors that can be used to receive user input, such as a keyboard, a mouse, a touchscreen, a microphone, and so on.

In some embodiments, communications systems 508 can include any suitable hardware, firmware, and/or software for communicating information over communication network 454 and/or any other suitable communication networks. For example, communications systems 508 can include one or more transceivers, one or more communication chips and/or chip sets, and so on. In a more particular example, communications systems 508 can include hardware, firmware and/or software that can be used to establish a Wi-Fi connection, a Bluetooth connection, a cellular connection, an Ethernet connection, and so on.

In some embodiments, memory 510 can include any suitable storage device or devices that can be used to store instructions, values, data, or the like, that can be used, for example, by processor 502 to present content using display 504, to communicate with server 452 via communications system(s) 508, and so on. Memory 510 can include any suitable volatile memory, non-volatile memory, storage, or any suitable combination thereof. For example, memory 510 can include RAM, ROM, EEPROM, one or more flash drives, one or more hard disks, one or more solid state drives, one or more optical drives, and so on. In some embodiments, memory 510 can have encoded thereon, or otherwise stored therein, a computer program for controlling operation of computing device 450. In such embodiments, processor 502 can execute at least a portion of the computer program to present content (e.g., visual alarms, user interfaces, graphics, tables), receive content from server 452, transmit information to server 452, and so on.

In some embodiments, server 452 can include a processor 512, a display 514, one or more inputs 516, one or more communications systems 518, and/or memory 520. In some embodiments, processor 512 can be any suitable hardware processor or combination of processors, such as a CPU, a GPU, and so on. In some embodiments, display 514 can include any suitable display devices, such as a computer monitor, a touchscreen, a television, and so on. In some embodiments, inputs 516 can include any suitable input devices and/or sensors that can be used to receive user input, such as a keyboard, a mouse, a touchscreen, a microphone, and so on.

In some embodiments, communications systems 518 can include any suitable hardware, firmware, and/or software for communicating information over communication network 454 and/or any other suitable communication networks. For example, communications systems 518 can include one or more transceivers, one or more communication chips and/or chip sets, and so on. In a more particular example, communications systems 518 can include hardware, firmware and/or software that can be used to establish a Wi-Fi connection, a Bluetooth connection, a cellular connection, an Ethernet connection, and so on.

In some embodiments, memory 520 can include any suitable storage device or devices that can be used to store instructions, values, data, or the like, that can be used, for example, by processor 512 to present content using display 514, to communicate with one or more computing devices 450, and so on. Memory 520 can include any suitable volatile memory, non-volatile memory, storage, or any suitable combination thereof. For example, memory 520 can include RAM, ROM, EEPROM, one or more flash drives, one or more hard disks, one or more solid state drives, one or more optical drives, and so on. In some embodiments, memory 520 can have encoded thereon a server program for controlling operation of server 452. In such embodiments, processor 512 can execute at least a portion of the server program to transmit information and/or content (e.g., data, a user interface) to one or more computing devices 450, receive information and/or content from one or more computing devices 450, receive instructions from one or more devices (e.g., a personal computer, a laptop computer, a tablet computer, a smartphone), and so on.

In some embodiments, subcutaneous EEG device 402 can include a processor 522, one or more inputs 524, one or more communications systems 526, and/or memory 528. In some embodiments, processor 522 can be any suitable hardware processor or combination of processors, such as a CPU, a GPU, and so on. In some embodiments, the one or more inputs 524 are generally configured to acquire data and can include relevant sensors or measurement devices to acquire the subcutaneous EEG data described above. Additionally or alternatively, in some embodiments, one or more inputs 524 can include any suitable hardware, firmware, and/or software for coupling to and/or controlling operations of a subcutaneous EEG device. In some embodiments, one or more portions of the one or more inputs 524 can be removable and/or replaceable.

Note that, although not shown, subcutaneous EEG device 402 can include any suitable inputs and/or outputs. For example, subcutaneous EEG device 402 can include, or be in communication with, input devices and/or sensors that can be used to receive user input, such as a keyboard, a mouse, a touchscreen, a microphone, a trackpad, a trackball, and so on. As another example, subcutaneous EEG device 402 can include, or be in communication with, any suitable display devices, such as a computer monitor, a touchscreen, a television, etc., one or more speakers, and so on.

In some embodiments, communications systems 526 can include any suitable hardware, firmware, and/or software for communicating information to computing device 450 (and, in some embodiments, over communication network 454 and/or any other suitable communication networks). For example, communications systems 526 can include one or more transceivers, one or more communication chips and/or chip sets, and so on. In a more particular example, communications systems 526 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 528 can include any suitable storage device or devices that can be used to store instructions, values, data, or the like, that can be used, for example, by processor 522 to control the one or more inputs 524, and/or receive data from the one or more inputs 524: present content (e.g., a user interface) using a display: communicate with one or more computing devices 450; and so on. Memory 528 can include any suitable volatile memory, non-volatile memory, storage, or any suitable combination thereof. For example, memory 528 can include RAM, ROM, EEPROM, one or more flash drives, one or more hard disks, one or more solid state drives, one or more optical drives, and so on. In some embodiments, memory 528 can have encoded thereon, or otherwise stored therein, a program for controlling operation of subcutaneous EEG device 402. In such embodiments, processor 522 can execute at least a portion of the program to transmit information and/or content (e.g., data) to one or more computing devices 450, receive information and/or content from one or more computing devices 450, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for predicting a seizure onset in electroencephalography (EEG) measurement data recorded with a subcutaneous EEG measurement device, the method comprising:
(a) recording subcutaneous EEG measurement data with the subcutaneous EEG device, wherein the subcutaneous EEG measurement data comprise EEG signals measured subcutaneously from a subject;
(b) accessing a trained machine learning algorithm with a computer system, wherein the trained machine learning algorithm has been trained on training data in order to predict a likelihood of seizure onset occurring within the EEG signals contained in the subcutaneous EEG measurement data;
(c) transmitting the subcutaneous EEG measurement data from the subcutaneous EEG device to the computer system; and
(d) applying the subcutaneous EEG measurement data to the trained machine learning algorithm with the computer system, generating output as an indication of seizure onset occurring in the subcutaneous EEG measurement data.

2. The method of claim 1, wherein the trained machine learning algorithm is trained on the training data using a multi-stage training process.

3. The method of claim 2, wherein the multi-stage training process includes training an initial machine learning algorithm on first training data and retraining the initial machine learning algorithm on second training data, generating output as the trained machine learning algorithm.

4. The method of claim 3, wherein the first training data comprise scalp-recorded EEG data and the second training data comprise subcutaneously recorded EEG data acquired from subjects.

5. The method of claim 4, wherein the initial machine learning algorithm is retrained using transfer learning on the second training data.

6. The method of claim 5, wherein the initial machine learning algorithm is trained using a multi-layer long short-term memory (LSTM) network.

7. The method of claim 6, wherein the multi-layer LSTM network comprises three LSTM network layers.

8. The method of claim 7, wherein the first and second LSTM network layers are non-trainable and the third LSTM network layer is trainable.

9. The method of claim 7, wherein the initial machine learning algorithm is trained using a recurrent neural network comprising at least one gated recurrent unit (GRU) layer.

10. The method of claim 7, wherein the initial machine learning algorithm is trained using a neural network having at least one convolutional layer.

11. The method of claim 7, wherein the initial machine learning algorithm is trained using a neural network having fully connected layers.

12. The method of claim 1, wherein the computer system is local to the subcutaneous EEG device.

13. The method of claim 1, wherein the computer system is physically separate from the subcutaneous EEG device.

14. The method of claim 1, further comprising generating an alarm to a user when the trained machine learning algorithm generates output indicating a seizure onset is likely to occur based on the subcutaneous EEG measurement data input to the trained machine learning algorithm.

15. The method of claim 14, wherein the alarm comprises an auditory alarm.

16. The method of claim 14, wherein the alarm comprises a visual alarm.

17. The method of claim 11, further comprising:
providing a user interface to the subject, via the computer system, that is configured to receive feedback on the indication of seizure onset occurring in the subcutaneous EEG measurement data;
receiving user feedback data, via the computer system, wherein the user feedback data indicates whether a seizure event occurred following the indication of seizure onset occurring in the subcutaneous EEG measurement data; and
adjusting a threshold for generating the alarm based on the user feedback data.

18. The method of claim 1, further comprising:
providing a user interface to the subject, via the computer system, that is configured to receive feedback on the indication of seizure onset occurring in the subcutaneous EEG measurement data;
receiving user feedback data, via the computer system, wherein the user feedback data indicates whether a seizure event occurred following the indication of seizure onset occurring in the subcutaneous EEG measurement data; and
retraining the machine learning algorithm based on the user feedback data.

19. The method of claim 18, wherein the machine learning algorithm is retrained based on the user feedback data using an active learning technique.
